(19) United States
(12) Patent Application Publication
(10) Pub. No.: US 2021/0082112 A1
(43) Pub. Date: Mar. 18, 2021

(54) IMAGE PROCESSING METHOD, ELECTRONIC DEVICE, AND STORAGE MEDIUM

(71) Applicant: BEIJING SENSETIME TECHNOLOGY DEVELOPMENT CO.,LTD., Beijing (CN)

(72) Inventors: Jiahui LI, Beijing (CN); Zhiqiang Hu, Beijing (CN); Wenji Wang, Beijing (CN); Yuxin Yao, Beijing (CN)

(21) Appl. No.: 17/104,264

(22) Filed: Nov. 25, 2020

Related U.S. Application Data
(63) Continuation of application No. PCT/CN2018/117862, filed on Nov. 28, 2018.

(30) Foreign Application Priority Data
Jul. 23, 2018 (CN) 201810814377.9

(51) Int. Cl. G06T 7/00; G06T 5/40; G06T 5/00; A61B 5/00; A61B 5/055

(52) U.S. Cl. CPC G06T 7/0012; G06T 5/40; G06T 5/009; A61B 5/0044; A61B 2576/023; G06T 2207/30048; G06T 2207/20084; G06T 2207/10088; A61B 5/055

(57) ABSTRACT

Embodiments of the present disclosure disclose an image processing method, an electronic device, and a storage medium. The method includes: converting an original image into a target image conforming to a target parameter; obtaining a target numerical index according to the target image; and performing, according to the target numerical index, timing prediction processing on the target image to obtain a timing state prediction result. Left ventricular function quantization can be implemented, the image processing efficiency is improved, and the prediction accuracy of a cardiac function index is improved.

### CROSS-REFERENCE TO RELATED APPLICATIONS

The present application is a continuation of International Application No. PCT/CN2018/117862, filed on Nov. 28, 2018, which claims priority to Chinese Patent Application No. 201810814377.9, filed on Jul. 23, 2018. The disclosures of International Application No. PCT/CN2018/117862 and Chinese Patent Application No. 201810814377.9 are hereby incorporated by reference in their entireties.

### BACKGROUND

Image processing is a technique in which an image is analyzed by a computer to achieve a desired result. The image processing generally refers to digital image processing. Digital images refer to a large two-dimensional array captured by a device such as an industrial camera, a camera, or a scanner. An element of the array is called a pixel, and the value thereof is called a grayscale value. The image processing plays an important role in many fields, especially in the medical field.

At present, left ventricular function quantification is the most important operation in the diagnostic procedure for diagnosing a heart disease. The left ventricular function quantification is still a difficult task due to the cardiac structure diversity and timing complexity of heartbeats of different patients. The specific goal of the left ventricular function quantification is to output specific indexes of each tissue of a left ventricle. In the past, when there was no computer assistance, a process for completing the above index calculation was that: a doctor manually circled the contours of a cardiac chamber and a myocardium on a medical image of a heart, calibrated the direction of a main axis, and then manually measured the specific indexes. The process wastes time and efforts, and the difference between the determinations of doctors is significant.

With the development and maturity of medical technology, a method for calculating an index by computer assistance has also been widely applied. In general, when a method for calculating an index after input and output pixels of an original image are segmented is used, segmentation is inaccurate at a fuzzy boundary portion of the image, and the doctor needs to intervene again to perform boundary correction before an accurate index can be obtained. Only the time for determining a myocardium and a cardiac chamber region can be saved. In the image processing of the left ventricular function quantification, the processing efficiency of the method is low, and the accuracy of the obtained index is not high.

### SUMMARY

The present disclosure relates to the field of image processing, and in particular, to an image processing method, an electronic device, and a storage medium.

Embodiments of the present disclosure provide an image processing method, an electronic device, and a storage medium.

A first aspect of the embodiments of the present disclosure provides an image processing method, including: converting an original image into a target image conforming to a target parameter; obtaining a target numerical index according to the target image; and performing, according to the target numerical index, timing prediction processing on the target image to obtain a timing state prediction result.

A second aspect of the embodiments of the present disclosure provides an electronic device, including: a memory storing processor-executable instructions; and a processor arranged to execute the stored processor-executable instructions to perform operations of: converting an original image into a target image conforming to a target parameter; obtaining a target numerical index according to the target image converted by the image converting module; and performing, according to the target numerical index, timing prediction processing on the target image to obtain a timing state prediction result.

A third aspect of the embodiments of the present disclosure provides a non-transitory computer readable storage medium having stored thereon computer-readable instructions that, when executed by a processor, cause the processor to implement an image processing method, the method including: converting an original image into a target image conforming to a target parameter; obtaining a target numerical index according to the target image; and performing, according to the target numerical index, timing prediction processing on the target image to obtain a timing state prediction result.

### BRIEF DESCRIPTION OF DRAWINGS

To describe the technical solutions in embodiments of the present disclosure or the prior art more clearly, the accompanying drawings required for describing the embodiments or the prior art are briefly introduced below.

FIG. 1 is a schematic flowchart of an image processing method disclosed in the embodiments of the present disclosure;

FIG. 2 is a schematic flowchart of another image processing method disclosed in the embodiments of the present disclosure;

FIG. 3 is a schematic structural diagram of an electronic device disclosed in the embodiments of the present disclosure;

FIG. 4 is a schematic structural diagram of another electronic device disclosed in the embodiments of the present disclosure.

### DETAILED DESCRIPTION

In order to enable a person skilled in the art to understand the solutions of the present invention better, the technical solutions of the embodiments of the present disclosure are clearly and fully described below with reference to the accompanying drawings in the embodiments of the present disclosure. Apparently, the described embodiments are merely some of the embodiments of the present invention rather than all the embodiments. Based on the embodiments of the present invention, all other embodiments obtained by a person of ordinary skill in the art without involving an inventive effort shall fall within the scope of protection of the present invention.

Terms “first”, “second”, and the like in the specification, claims, and the drawings in the embodiments of the present disclosure are used for distinguishing between different objects, rather than describing a specific order. In addition, terms “include” and “have” and any deformation thereof aim at covering non-exclusive “comprising”, for example, a process, method, system, product, or device comprising a series of operations or units is not limited to the operations or units that have been listed, but optionally also include other operations or units that are not listed, or optionally also include other operations or units inherent to the process, method, product, or device.

Reference to “embodiment” herein means that a particular feature, structure, or characteristic described in combination with the embodiments may be included in at least one embodiment of the present invention. The phrases appearing in various places in the specification do not necessarily refer to the same embodiment, and are not independent or alternative embodiments that are mutually exclusive. A person skilled in the art explicitly and implicitly understands that the embodiments described herein may be combined with other embodiments.

The electronic device involved in the embodiments of the present disclosure may allow access by a plurality of other terminal devices. The electronic device includes a terminal device. In specific implementations, the terminal device includes, but is not limited to, other portable devices such as a mobile phone, a laptop computer, or a tablet computer having a touch-sensitive surface (e.g. a touch screen display and/or a touch pad). It should also be understood that in some embodiments, the device is not a portable communication device but a desktop computer having a touch-sensitive surface (e.g. a touch screen display and/or a touch pad).

The concept of deep learning in the embodiments of the present disclosure derives from the research of an artificial neural network. A multilayer perceptron with multiple hidden layers is a deep learning structure. The deep learning combines low-level features to form more abstract high-level representation attribute categories or features to discover distributed feature representations of data.

The deep learning is a method based on representation learning of data in machine learning. An observation value (e.g. an image) may be represented in a plurality of manners, such as a vector of each pixel intensity value, or more abstractly represented as a series of edges, regions with a particular shape, and the like. Learning a task (e.g. face recognition or facial expression recognition) from instances is easier using some specific representation methods. The advantage of the deep learning is to replace manual feature acquisition with unsupervised or semi-supervised feature learning and a hierarchical feature extraction efficient algorithm. The deep learning is a new field in machine learning research, and the motivation thereof lies in the establishment and simulation of a neural network of the human brain for analysis and learning. It simulates the mechanism of the human brain to interpret data such as images, sounds, and texts.

The embodiments of the present disclosure are introduced in detail below.

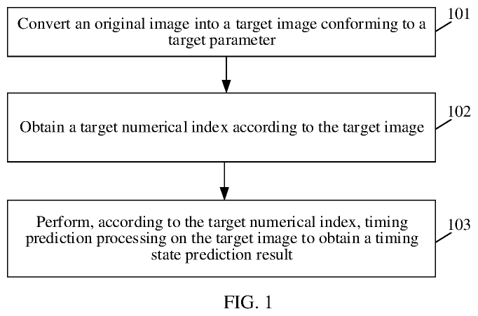

Referring to FIG. 1, FIG. 1 is a schematic flowchart of an image processing method disclosed in the embodiments of the present disclosure. As shown in FIG. 1, the image processing method may be executed by the electronic device, and includes the following operations 101 to 103.

At 101, an original image is converted into a target image conforming to a target parameter.

Before the image processing is performed by means of a deep learning model, the original image is first subjected to image pre-processing and converted into the target image conforming to the target parameter, and then operation 102 is performed. The main purpose of the image pre-processing is to eliminate irrelevant information in the image, restore useful and real information, enhance the detectability of relevant information, and simplify data to the greatest extent, thereby improving the reliability of feature extraction, image segmentation, matching, and recognition.

The original image mentioned in the embodiments of the present disclosure may be a heart image obtained by various medical image devices, and has diversity, which is embodied in the image as the diversity of macro features such as contrast and brightness. The number of the original image in the embodiments of the present disclosure may be one or more. If the pre-processing is not performed according to a conventional technique, and a new image just possesses macro features that have not been learned before, the model may have a large error.

The target parameter may be understood as a parameter describing an image feature, i.e., a prescribed parameter for making the original images in a unified style. For example, the target parameter may include parameters for describing a feature such as image resolution, image grayscale, and image size, and the target image parameters may be stored in the electronic device. In the embodiments of the present disclosure, a parameter describing a range of image grayscale values may be selected.

As an example, the manner for obtaining the target image conforming to the target parameter may include: performing histogram equalization processing on the original image to obtain the target image of which a grayscale value satisfies a target dynamic range.

If the pixels of an image occupy a large number of greyscale levels and are distributed uniformly, such an image tends to have a high contrast and a variable greyscale tone. The histogram equalization mentioned in the embodiments of the present disclosure is a transformation function capable of automatically achieving this effect only by inputting histogram information of the image, and the basic idea thereof is to broaden the greyscale level having a large number of pixels in the image and compress the greyscale level having a small number of pixels in the image, thereby expanding the dynamic range of pixel values, improving changes of the contrast and grayscale tone to make the image clearer.

The embodiments of the present disclosure may pre-process the original image by using a histogram equalization method to reduce the diversity between the images. The target dynamic range for the grayscale values may be pre-stored in the electronic device, and may be set by a user in advance, and when the histogram equalization processing is performed on the original image, the grayscale values of the image satisfy the target dynamic range (for example, all the original images are stretched to a maximum grayscale dynamic range), that is, the target image is obtained.

By pre-processing the original image, the diversity thereof may be reduced. After a unified and clear target image is obtained by the histogram equalization, the subsequent image processing operations are then performed, and the deep learning model can provide a more stable determination.

In one optional example, operation 101 may be executed by a processor by invoking a corresponding instruction stored in a memory, or may be executed by an image converting module 310 run by the processor.

At 102, a target numerical index is obtained according to the target image. As an implementation mode, a plurality of indexes of left ventricular function quantification may be obtained by means of an index predicting module. The index predicting module in the embodiments of the present disclosure may execute a deep learning network model to obtain a target numerical index, and the deep learning network model may be, for example, a deep layer aggregation network model.

The deep learning network used in the embodiments of the present disclosure is called a Deep Layer Aggregation Network (DLANet), which is also called a deep layer aggregation structure. A standard system structure is extended by deeper aggregation to aggregate the information of each layer better, and deep layer aggregation combines feature hierarchical structures in an iterative and hierarchical manner, so that the network has high accuracy and fewer parameters. Using a tree construction to replace a linear construction of a previous architecture implements, instead of linear compression, logarithmic level compression of a gradient backhaul length of the network so that the learned features have better descriptive capability, which may effectively improve the prediction accuracy of the numerical index.

By means of the deep layer aggregation network models, the target image is processed to obtain a corresponding target numerical index. The specific goal of left ventricular function quantification is to output specific indexes of each tissue of a left ventricle, generally including cardiac chamber area, myocardial area, cardiac chamber diameters at every 60 degrees, and myocardium thicknesses at every 60 degrees, which have 1, 1, 3, and 6 numerical output indexes, respectively, a total of 11 numerical output indexes. Specifically, the original image may be Magnetic Resonance Imaging (MRI), and for cardiovascular diseases, not only may anatomical changes of various chambers, large blood vessels, and valves be observed, but also ventricular analysis for qualitative and semi-quantitative diagnoses is made, and a plurality of section images having a high spatial resolution may be made so as to display the whole picture of a heart and a lesion and their relationship to the surrounding structure.

The target numerical index may include any one or more of the following: cardiac chamber area, myocardial area, cardiac chamber diameters at every 60 degrees, and myocardium thicknesses at every 60 degrees. By using the deep layer aggregation network models, after a cardiac MRI median slice of a patient is obtained, physical indexes such as the cardiac chamber area, myocardial area, the diameter of the cardiac chamber, and the thickness of myocardium of the heart in the image may be calculated for subsequent medical treatment analyses.

In addition, in the specific implementation process of the operation, the DLANet involved may be trained by means of a large number of original images. When the network model is trained using a data set of the original images, the pre-processing operation may still be performed first, that is, the histogram equalization method may be used first to reduce the diversity between the original images, so that the learning and determination accuracy of the model are improved.

In one optional example, operation 102 may be executed by a processor by invoking a corresponding instruction stored in a memory, or may be executed by an index predicting module 320 run by the processor.

At 103, timing prediction processing is performed on the target image according to the target numerical index to obtain a timing state prediction result.

After the target numerical index is obtained, the timing state prediction of the systole and diastole of the heart may be performed. In general, a circular network is used for predicting the state, and the determination is mainly made by using cardiac chamber area values. In the embodiments of the present disclosure, when the timing state prediction of the systole and diastole of the heart is made, a parameterless sequence prediction policy may be used for timing prediction, and the parameterless sequence prediction policy refers to a prediction policy that does not introduce any additional parameters.

Specifically, for heartbeat image data of a patient, multiple image frames may be obtained. First, the cardiac chamber area value of each image frame is predicted by deep layer aggregation network models to obtain a prediction of the cardiac chamber area value of each frame as a prediction point; second, a multi-power polynomial curve may be used to fit the prediction points; and finally, a highest frame and a lowest frame of a regression curve are taken to determine the systole and diastole of the heart.

In one optional example, operation 103 may be executed by a processor by invoking a corresponding instruction stored in a memory, or may be executed by a state predicting module 330 run by the processor.

Specifically, obtaining the target numerical index in operation 102 may include:

respectively obtaining M predicted cardiac chamber area values of M target image frames; and

operation 103 may include:

(1) fitting the M predicted cardiac chamber area values by using a polynomial curve to obtain a regression curve;

(2) obtaining a highest frame and a lowest frame of the regression curve to obtain a determination interval for determining whether the cardiac state is a systolic state or a diastolic state; and

(3) determining the cardiac state according to the determination interval, where M is an integer greater than 1.

Data fitting, also called curve fitting, commonly known as the pull curve, is a representation manner in which the existing data is substituted into a numerical expression by means of a mathematical method. For scientific and engineering problems, several discrete data is obtained by a method such as sampling and experiments, according to these data, it is often desirable to obtain a continuous function (that is, a curve) or a more dense discrete equation that matches the known data, and the process is called fitting.

In a machine learning algorithm, a linear model based on a nonlinear function of data is common. The method may perform operations as efficient as the linear model, and the model may be applied to a wider range of data.

The M target image frames may cover at least one heartbeat cycle, i.e., performing prediction for multiple image frames acquired in one heartbeat cycle, and the determination of the cardiac state may be performed more accurately. For example, 20 target image frames in one heartbeat cycle of the patient may be obtained. First, prediction processing is performed on each of the 20 target image frames by means of the DLANet in operation 102 to obtain a predicted cardiac chamber area value corresponding to each target image frame so as to obtain 20 prediction points. Then, an 11th power polynomial curve is used for fitting the 20 prediction points, and finally a highest frame and a lowest frame of the regression curve are taken to calculate the determination interval. For example, the frames between (the highest point, the lowest point] are determined to be in systolic state 0 and the frames between (the lowest point, the highest point] are determined to be in diastolic state 1, so that the timing state prediction of the systole and diastole may be obtained, which facilitates subsequent medical analyses, and assists a doctor to carry out targeted treatment for pathological conditions.

The timing network (Long Short Term Memory Networks, LSTM) in the embodiments of the present disclosure refers to a special concept mode that describes the state of a system and a conversion mode thereof by means of two basic concepts of state and conversion. For the prediction of the systolic and diastolic states, the use of the parameterless sequence prediction policy may achieve higher determination accuracy and solve a problem of discontinuous prediction than a timing network generally used. In a general method, the prediction of the systolic and diastolic states of the heart is performed by means of the timing network. In the use of the manner of the timing network, the determining such as “0-1-0-1” (1 means systole, 0 means diastole) is inevitable, which causes the problem of discontinuous prediction, but in fact, the heart must be systolic for a whole segment and is diastolic for a whole segment in one cycle, and no frequent state changes occur. Using the parameterless sequence prediction policy to replace the timing network fundamentally solves the problem of discontinuous prediction, the determination for unknown data is more stable, and the robust of the policy is stronger because there are no additional parameters, and higher prediction accuracy than when there is a timing network may be obtained. The so-called robust refers to a characteristic of controlling the system to maintain some other performances under a certain (structure and size) parameter perturbation, means robust and strong in English, and is the key to the survival of the system in abnormal and dangerous situations. For example, if an input error, disk failure, network overload, or an intentional attack occurs in computer software, crashing and a dead halt still do not occur, which is the robust of the software.

By converting an original image into a target image conforming to a target parameter; obtaining a target numerical index according to the target image; and performing, according to the target numerical index, timing prediction processing on the target image to obtain a timing state prediction result, the embodiments of the present invention can implement left ventricular function quantization, improve the image processing efficiency, reduce labor consumption and errors caused by manual participation in general processing, and improve the prediction accuracy of the cardiac function index.

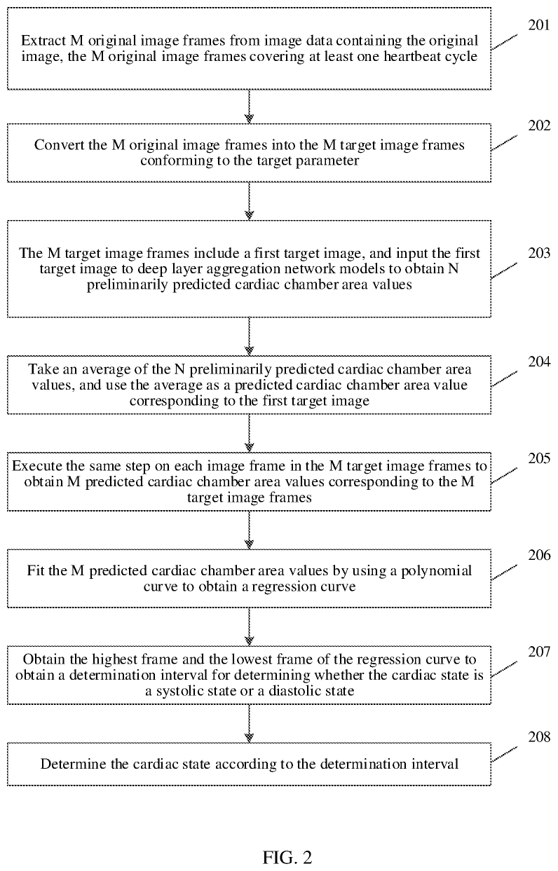

Referring to FIG. 2, FIG. 2 is a schematic flowchart of another image processing method disclosed in the embodiments of the present disclosure. FIG. 2 is further obtained on the basis of FIG. 1. The main body executing operations of the embodiments of the present disclosure may be an electronic device for medical image processing. As shown in FIG. 2, the image processing method includes the following operations 201 to 208.

At 201, M original image frames are extracted from image data containing the original image, where the M original image frames cover at least one heartbeat cycle.

The M target image frames may cover at least one heartbeat cycle, i.e., performing prediction for multiple image frames acquired in one heartbeat cycle, and the determination of the cardiac state is performed more accurately.

At 202, the M original image frames are converted into the M target image frames conforming to the target parameter.

M is an integer greater than 1, and optionally, M may be 20, i.e., obtaining 20 target image frames in one heartbeat cycle of the patient. For the image pre-processing process of operation 202, reference may be made to the specific description in operation 101 of the embodiments shown in FIG. 1, and details are not described herein again.

At 203, the M target image frames include a first target image, and the first target image is input to the N deep layer aggregation network models to obtain N preliminarily predicted cardiac chamber area values.

For convenience of description and understanding, one frame among the M target image frames, i.e., the first target image is taken as an example for specific description. The number of the deep layer aggregation network models in the embodiments of the present disclosure may be N, where N is an integer greater than 1. According to one or more embodiment of the present disclosure, the N deep layer aggregation network models are obtained by subjecting training data to cross-validation training.

The cross-validation mentioned in the embodiments of the present disclosure is mainly used in modeling applications, such as Principal Component Analysis (PCR) and Partial Least Squares Regression (PLS) modeling. Specifically, it can be understood that in a given modeling sample, most of the samples are taken to build a model, a small part of the samples is used for prediction by using the newly built model, and prediction errors of the small part of the samples are obtained, and the sum of their square plus are recorded.

In the embodiments of the present disclosure, a cross-validation training method may be used. Optionally, five-cross-validation training may be selected, the existing training data is subjected to the five-cross-validation training to obtain five models (the deep layer aggregation network models), and when verification, a whole data set is used for embodying algorithm results. Specifically, when the data is divided into five parts, first, a grayscale histogram of each original image after being pre-processed and cardiac function indexes (which may be the 11 indexes above) may be extracted and connected as a descriptor of the target image; then, a mean value K is used for unsupervisedly dividing the training data into five categories, then each of the five categories of training data is divided into five equal parts, each data is taken from one of the five equal parts of each category of data (four parts may be used for training and one part may be used for verification), and by means of the operations above, the five models may widely learn the characteristics of each type of data when five-cross-validation, thereby improving the robust of the models.

Moreover, compared with the random division in conventional image processing, for the five-cross-validation training, the obtained models are less likely to exhibit extreme deviations that are caused by unbalanced data training.

After the N preliminarily predicted cardiac chamber area values of the first target image are obtained by means of the N models, operation 204 may be performed.

At 204, an average of the N preliminarily predicted cardiac chamber area values is taken, and the average is used as a predicted cardiac chamber area value corresponding to the first target image.

At 205, the same operation is executed on each of the M target image frames to obtain M predicted cardiac chamber area values corresponding to the M target image frames.

Operations 203 and 204 are processing for one target image frame, and the same operation may be executed on the M target image frames to obtain the predicted cardiac chamber area value corresponding to each target image frame, and the processing of the M target image frames may be performed synchronously, so as to improve processing efficiency and accuracy.

By means of the five-cross-validation training method, when new data (a new original image) is predicted, five prediction results of the cardiac chamber area may be obtained by means of the five models, and then averaged to obtain a final regression prediction result. This prediction result may be used for operation 206 and timing determination processes after operation 206. By means of multi-model aggregation, the accuracy of the prediction index is improved.

At 206, the M predicted cardiac chamber area values are fitted by using a polynomial curve to obtain a regression curve.

At 207, a highest frame and a lowest frame of the regression curve are obtained to obtain a determination interval for determining whether the cardiac state is a systolic state or a diastolic state.

At 208, the cardiac state is determined according to the determination interval.

For operations 206-208, reference may be made to the specific descriptions of (1)-(3) in operation 103 of the embodiments shown in FIG. 1, and details are not described herein again.

The embodiments of the present disclosure are applicable to clinical medical assistance diagnoses. After obtaining a cardiac MRI median slice of a patient, the doctor needs to calculate physical indexes such as the cardiac chamber area, myocardial area, a diameter of a cardiac chamber, and a thickness of myocardium of the heart in the image. The method may be used to quickly obtain more accurate determination of the indexes (which may be completed in 0.2 second) without the need of time-consuming and laborious manual measurement calculations on the image, so as to assist the doctor to determine a disease according to the physical indexes of the heart.

By extracting M original image frames from image data containing the original image, the M original image frames covering at least one heartbeat cycle; then converting the M original image frames into the M target image frames conforming to the target parameter, where the M target image frames include a first target image; inputting the first target image to the N deep layer aggregation network models to obtain N preliminarily predicted cardiac chamber area values; then taking an average of the N preliminarily predicted cardiac chamber area values and using the average as a predicted cardiac chamber area value corresponding to the first target image, and executing the same operation on each of the M target image frames to obtain M predicted cardiac chamber area values corresponding to the M target image frames; fitting the M predicted cardiac chamber area values by using a polynomial curve to obtain a regression curve; obtaining a highest frame and a lowest frame of the regression curve to obtain a determination interval for determining whether the cardiac state is a systolic state or a diastolic state; and further determining the cardiac state according to the determination interval, the embodiments of the present disclosure implement left ventricular function quantization, improve the image processing efficiency, reduce labor consumption and errors caused by manual participation in general processing, and improve the prediction accuracy of a cardiac function index.

The above description mainly introduces the solutions of the embodiments of the present disclosure with the perspective of executing the processes from a method side. It can be understood that in order to implement the above functions, the electronic device includes corresponding hardware structures and/or software modules for executing the functions. A person skilled in the art shall be easily aware that the present invention can be implemented by hardware or a combination of hardware and computer software in combination with the units and arithmetic operations of the examples described in the embodiments disclosed herein. Whether a certain function is implemented by hardware or a manner of computer software driving hardware depends on the particular applications and design constraint conditions of the technical solutions. A person skilled in the art may use different methods to implement the described functions for each particular application, but it should not be considered that this implementation goes beyond the scope of the present invention.

The embodiments of the present disclosure may divide functional modules of the electronic device according to the foregoing method examples. For example, each functional module may be divided according to each function, or two or more functions may also be integrated into one processing module. The integrated module may be implemented in the form of hardware or in the form of a software functional module. It should be noted that the division of the modules in the embodiments of the present disclosure is schematic, and is merely a logical function division, and there may be another division manner in actual implementation.

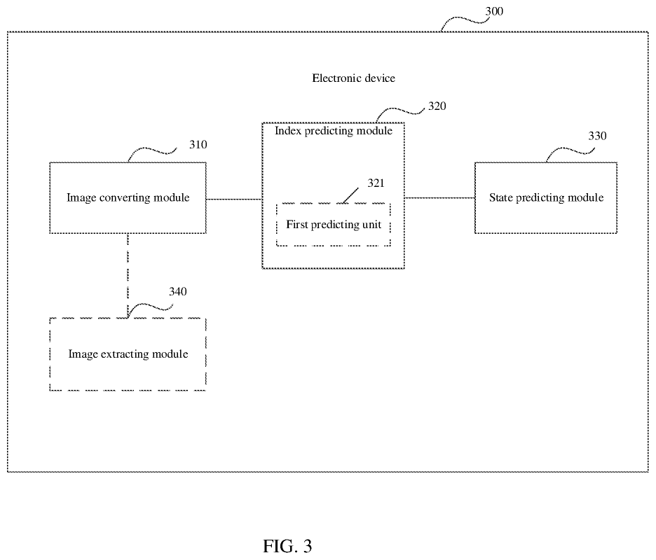

Referring to FIG. 3, FIG. 3 is a schematic structural diagram of an electronic device disclosed in the embodiments of the present disclosure. As shown in FIG. 3, the electronic device 300 includes: an image converting module 310, an index predicting module 320, and a state predicting module 330, where:

the image converting module 310 is configured to convert an original image into a target image conforming to a target parameter;

the index predicting module 320 is configured to obtain a target numerical index according to the target image converted by the image converting module 310; and

the state predicting module 330 is configured to perform timing prediction processing on the target image according to the target numerical index obtained by the index predicting module 320 to obtain a timing state prediction result.

According to one or more embodiment of the present disclosure, the index predicting module 320 is configured to: perform the timing prediction processing on the target image by using a parameterless sequence prediction policy to obtain the timing state prediction result.

According to one or more embodiment of the present disclosure, the index predicting module 320 is configured to: obtain the target numerical index according to the target image and deep layer aggregation network models.

According to one or more embodiment of the present disclosure, the original image is cardiac magnetic resonance imaging; and

the target numerical index includes at least one of cardiac chamber area, myocardial area, cardiac chamber diameters at every 60 degrees, and myocardium thicknesses at every 60 degrees.

According to one or more embodiment of the present disclosure, the index predicting module 320 includes a first predicting unit 321 and the first predicting unit 321 is configured to: respectively obtain M predicted cardiac chamber area values of M target image frames; and

The state predicting module 330 is configured to: fit the M predicted cardiac chamber area values by using a polynomial curve to obtain a regression curve; obtain a highest frame and a lowest frame of the regression curve to obtain a determination interval for determining whether the cardiac state is a systolic state or a diastolic state; and determine the cardiac state according to the determination interval, M being an integer greater than 1.

According to one or more embodiment of the present disclosure, the electrode device 300 further includes an image extracting module 340 configured to extract M original image frames from image data containing the original image, the M original image frames covering at least one heartbeat cycle; and

the image converting module 310 is configured to convert the M original image frames into the M target image frames conforming to the target parameter.

According to one or more embodiment of the present disclosure, the number of the deep layer aggregation network models of the index predicting module 320 is N, and the N deep layer aggregation network models are obtained by subjecting training data to cross-validation training, N being an integer greater than 1.

According to one or more embodiment of the present disclosure, the M target image frames include a first target image, and the index predicting module 320 is configured to: input the first target image to the N deep layer aggregation network models to obtain N preliminarily predicted cardiac chamber area values; and

the first predicting unit 321 is configured to: take an average of the N preliminarily predicted cardiac chamber area values and use the average as a predicted cardiac chamber area value corresponding to the first target image, and execute the same operation on each of the M target image frames to obtain M predicted cardiac chamber area values corresponding to the M target image frames.

According to one or more embodiment of the present disclosure, the image converting module 310 is configured to: perform histogram equalization processing on the original image to obtain the target image of which a grayscale value satisfies a target dynamic range.

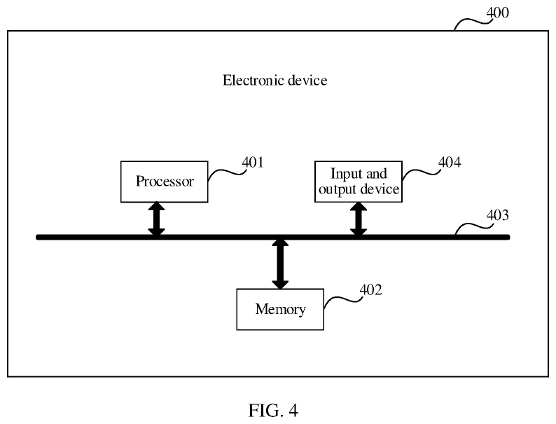

When the electronic device 300 shown in FIG. 3 is implemented, the electronic device 300 may convert an original image into a target image conforming to a target parameter; obtain a target numerical index according to the target image; and perform, according to the target numerical index, timing prediction processing on the target image to obtain a timing state prediction result. Left ventricular function quantization can be implemented, the image processing efficiency is improved, labor consumption and errors caused by manual participation in general processing is reduced, and the prediction accuracy of a cardiac function index is improved. Referring to FIG. 4, FIG. 4 is a schematic structural diagram of an electronic device disclosed in the embodiments of the present disclosure. As shown in FIG. 4, the electronic device 400 includes a processor 401 and a memory 402, where the memory 402 is configured to store one or more programs, the one or more programs are configured to be executed by the processor 401, and the program(s) includes for executing the method of the embodiments of the present disclosure.

The electronic device 400 may further include a bus 403, the processor 401 and the memory 402 may be connected to each other by means of the bus 403, and the bus 403 may be a Peripheral Component Interconnect (PCI) bus or an Extended Industry Standard Architecture (EISA) bus. The bus 403 may be divided into an address bus, a data bus, a control bus, and the like. For ease of representation, only one thick line is shown in FIG. 4, but it does not mean that there is only one bus or one type of bus. The electronic device 400 may further include an input and output device 404, and the input and output device 404 may include a display screen, such as a liquid crystal display screen. The memory 402 is configured to store one or more programs containing instructions; the processor 401 is configured to invoke the instructions stored in the memory 402 to execute some or all of the operations of the method mentioned in the embodiments of the forgoing FIG. 1 and FIG. 2. The processor 401 may correspondingly implement the functions of the modules in the electronic device 300 in FIG. 3.

As an example, when the processor 401 executes a program stored in the memory 402, it is configured to execute the operations of: converting an original image into a target image conforming to a target parameter; obtaining a target numerical index according to the target image; and performing, according to the target numerical index, timing prediction processing on the target image to obtain a timing state prediction result. Left ventricular function quantization can be implemented, the image processing efficiency is improved, labor consumption and errors caused by manual participation in general processing is reduced, and the prediction accuracy of a cardiac function index is improved.

The embodiments of the present disclosure further provide a computer storage medium, where the computer storage medium is configured to store a computer program for electronic data exchange, and the computer program causes a computer to execute some or all of the operations of any one of the image processing methods described in the foregoing method embodiments.

It should be noted that, for the sake of simple description, the foregoing method embodiments are all expressed as a series of action combinations, but a person skilled in the art shall be aware that the present invention is not limited by the described action sequence because certain operations may be performed in other sequences or simultaneously in accordance with the present invention. In addition, a person killed in the art shall also be aware that the embodiments described in the specification are all preferred embodiments, and the actions and modules involved are not necessarily required for the present invention.

In the forgoing embodiments, description of the embodiments all have their own focuses, and for parts that are not described in detail in one embodiment, refer to the related description in other embodiments.

It should be understood that the disclosed apparatus in the several embodiments provided by the present disclosure may be implemented by other modes. For example, the apparatus embodiments described above are merely schematic. For example, the division of the modules (or units) is merely a logical function division, and there may be another division manner in actual implementation. For example, a plurality of modules or components may be combined or integrated into another system, or some features may be ignored or not executed. In addition, the displayed or discussed mutual couplings or direct couplings or communication connections may be implemented by means of some interfaces. The indirect couplings or communication connections between the apparatuses or modules may be implemented in electronic or other forms.

The modules described as separate members may or may not be physically separate, and the members displayed as modules may or may not be physical modules, that is, may be located in one position, or may be distributed on a plurality of network modules. A part of or all of the modules may be selected according to actual needs to achieve the purposes of the solutions of the embodiments.

In addition, the functional modules in the embodiments of the present invention may be integrated into one processing module, or each of the modules may exist alone physically, or two or more modules are integrated into one module. The integrated module may be implemented in a form of hardware, or may also be implemented in a form of a software functional module.

When the integrated module is implemented in a form of a software functional module and sold or used as an independent product, the integrated module may be stored in a computer readable memory. Based on such an understanding, the technical solutions of the present invention or a part thereof contributing to the prior art or all or some of the technical solutions may be essentially embodied in the form of a software product. The computer software product is stored in one memory and includes several instructions so that one computer device (which may be a personal computer, a server, a network device, or the like) executes all or some of operations of the method in the embodiments of the present invention. Moreover, the preceding memory includes: media having program codes stored such as a USB flash disk, a Read-only Memory (ROM), a Random Access Memory (RAM), a mobile hard disk drive, a floppy disk, and an optical disk.

A person of ordinary skill in the art may understand that all or some of operations of the methods of the foregoing embodiments may be completed by a program to instruct related hardware, and the program may be stored in a computer readable memory, and the memory may include: a flash drive, an ROM, an RAM, a magnetic disk, or an optical disk.

The embodiments of the present disclosure are described in detail above, the principles and implementation modes of the present invention are described herein by using specific examples, and the explanation of the embodiments is only used for helping to understand the method of the present invention and its core idea. Moreover, for a person of ordinary skill in the art, in accordance with the idea of the present invention, there are changes in the specific implementation modes and application scope. In conclusion, the content of the specification shall not be understood to be restriction of the present invention.

1. An image processing method, comprising:
converting an original image into a target image conforming to a target parameter;
obtaining a target numerical index according to the target image; and
performing, according to the target numerical index, timing prediction processing on the target image to obtain a timing state prediction result.

2. The image processing method according to claim 1, wherein the performing timing prediction processing on the target image to obtain a timing state prediction result comprises:
performing the timing prediction processing on the target image by using a parameterless sequence prediction policy to obtain the timing state prediction result.

3. The image processing method according to claim 1, wherein the obtaining a target numerical index according to the target image comprises: obtaining the target numerical index according to the target image and deep layer aggregation network models.

4. The image processing method according to claim 1, wherein the original image is a cardiac image obtained using magnetic resonance imaging, and
the target numerical index comprises at least one of: cardiac chamber area, myocardial area, cardiac chamber diameters at every 60 degrees, and myocardium thicknesses at every 60 degrees.

5. The image processing method according to claim 1, wherein the obtaining a target numerical index comprises:
respectively obtaining M predicted cardiac chamber area values of M target image frames; and
the performing, according to the target numerical index, the timing prediction processing on the target image by using a parameterless sequence prediction policy to obtain the timing state prediction result comprises:
fitting the M predicted cardiac chamber area values by using a polynomial curve to obtain a regression curve;
obtaining a highest frame and a lowest frame of the regression curve to obtain a determination interval for determining whether a cardiac state is a systolic state or a diastolic state; and
determining the cardiac state according to the determination interval, M being an integer greater than 1.

6. The image processing method according to claim 5, further comprising: before the converting an original image into a target image conforming to a target parameter,
extracting M original image frames from image data containing the original image, the M original image frames covering at least one heartbeat cycle; and
the converting an original image into a target image conforming to a target parameter comprises:
converting the M original image frames into the M target image frames conforming to the target parameter.

7. The image processing method according to claim 5, further comprising:
inputting the target image to deep layer aggregation network models to obtain the target numerical index,
wherein a number of the deep layer aggregation network models is N, and the N deep layer aggregation network models are obtained by subjecting training data to cross-validation training, N being an integer greater than 1.

8. The image processing method according to claim 7, wherein the M target image frames comprise a first target image, and the inputting the target image to the deep layer aggregation network models to obtain the target numerical index comprises:
inputting the first target image to the N deep layer aggregation network models to obtain N preliminarily predicted cardiac chamber area values; and
the respectively obtaining M predicted cardiac chamber area values of M target image frames comprises:
taking an average of the N preliminarily predicted cardiac chamber area values and using the average as a predicted cardiac chamber area value corresponding to the first target image, and executing same operations on each of the M target image frames to obtain the M predicted cardiac chamber area values corresponding to the M target image frames.

9. The image processing method according to claim 1, wherein the converting an original image into a target image conforming to a target parameter comprises:
performing histogram equalization processing on the original image to obtain the target image of which a grayscale value satisfies a target dynamic range.

10. An electronic device, comprising:
a memory storing processor-executable instructions; and
a processor arranged to execute the stored processor-executable instructions to perform operations of:
converting an original image into a target image conforming to a target parameter;
obtaining a target numerical index according to the target image; and
performing, according to the target numerical index, timing prediction processing on the target image to obtain a timing state prediction result.

11. The electronic device according to claim 10, wherein the performing timing prediction processing on the target image to obtain a timing state prediction result comprises: performing the timing prediction processing on the target image by using a parameterless sequence prediction policy to obtain the timing state prediction result.

12. The electronic device according to claim 10, wherein the obtaining a target numerical index according to the target image comprises: obtaining the target numerical index according to the target image and deep layer aggregation network models.

13. The electronic device according to claim 10, wherein the original image is cardiac magnetic resonance imaging, and
the target numerical index comprises at least one of: cardiac chamber area, myocardial area, cardiac chamber diameters at every 60 degrees, and myocardium thicknesses at every 60 degrees.

14. The electronic device according to claim 10, wherein the obtaining a target numerical index comprises:
respectively obtaining M predicted cardiac chamber area values of M target image frames; and
the performing, according to the target numerical index, the timing prediction processing on the target image by using a parameterless sequence prediction policy to obtain the timing state prediction result comprises:
fitting the M predicted cardiac chamber area values by using a polynomial curve to obtain a regression curve;
obtaining a highest frame and a lowest frame of the regression curve to obtain a determination interval for determining whether a cardiac state is a systolic state or a diastolic state; and
determining the cardiac state according to the determination interval, M being an integer greater than 1.

15. The electronic device according to claim 14, wherein the processor is arranged to execute the stored processor-executable instructions to further perform an operation of: before the converting an original image into a target image conforming to a target parameter,
extracting M original image frames from image data containing the original image, the M original image frames covering at least one heartbeat cycle; and
the converting an original image into a target image conforming to a target parameter comprises:
converting the M original image frames into the M target image frames conforming to the target parameter.

16. The electronic device according to claim 14, wherein the processor is arranged to execute the stored processor-executable instructions to further perform an operation of:
inputting the target image to deep layer aggregation network models to obtain the target numerical index,
wherein a number of the deep layer aggregation network models is N, and the N deep layer aggregation network models are obtained by subjecting training data to cross-validation training, N being an integer greater than 1.

17. The electronic device according to claim 16, wherein the M target image frames comprise a first target image, and the inputting the target image to the deep layer aggregation network models to obtain the target numerical index comprises:
inputting the first target image to the N deep layer aggregation network models to obtain N preliminarily predicted cardiac chamber area values; and
the respectively obtaining M predicted cardiac chamber area values of M target image frames comprises:
taking an average of the N preliminarily predicted cardiac chamber area values and use the average as a predicted cardiac chamber area value corresponding to the first target image, and executing same operations on each of the M target image frames to obtain the M predicted cardiac chamber area values corresponding to the M target image frames.

18. The electronic device according to claim 10, wherein the converting an original image into a target image conforming to a target parameter comprises:
performing histogram equalization processing on the original image to obtain the target image of which a grayscale value satisfies a target dynamic range.

19. A non-transitory computer readable storage medium having stored thereon computer-readable instructions that, when executed by a processor, cause the processor to implement an image processing method, the method comprising:
converting an original image into a target image conforming to a target parameter;
obtaining a target numerical index according to the target image; and
performing, according to the target numerical index, timing prediction processing on the target image to obtain a timing state prediction result.

20. The non-transitory computer readable storage medium according to claim 19, wherein the performing timing prediction processing on the target image to obtain a timing state prediction result comprises:
performing the timing prediction processing on the target image by using a parameterless sequence prediction policy to obtain the timing state prediction result.
